(19) United States
(12) Patent Application Publication
(10) Pub. No.: US 2026/0026959 A1
(43) Pub. Date: Jan. 29, 2026

(54) OSTOMY BODY FITMENT

(71) Applicant: ConvaTec Limited, Flintshire (GB)

(72) Inventor: Jost Siebert, Soborg (DK)

(21) Appl. No.: 19/343,029

(22) Filed: Sep. 29, 2025

Related U.S. Application Data
(63) Continuation of application No. PCT/US2024/021887, filed on Mar. 28, 2024.
(60) Provisional application No. 63/493,079, filed on Mar. 30, 2023.

(30) Foreign Application Priority Data
Jul. 12, 2023 (GB) 2310678.4

(51) Int. Cl. A61F 5/443; A61F 5/44

(52) U.S. Cl. CPC A61F 5/443; A61F 5/4404

(57) ABSTRACT

An ostomy body fitment for forming a seal between an ostomy appliance and a stoma of an ostomate. The ostomy fitment has a pair of adhesive layers and a substrate layer arranged between the adhesive layers. The adhesive layers each have a first hue and the substrate layer has a second hue. The colour of the first hue is complementary to the colour of the second hue. At least one of the adhesive layers may be translucent. A surface of the substrate layer may comprise an image. The image may be visible through a translucent adhesive layer of the pair of adhesive layers. The image may relate to one or more procedural steps in the utilization of the ostomy body fitment.

### TECHNICAL FIELD OF THE INVENTION

The present invention provides an ostomy body fitment for forming a seal between an ostomy appliance and a stoma of an ostomate.

### BACKGROUND TO THE INVENTION

Individuals that cannot pass waste naturally due to illness, injury or disease may require a colostomy, ileostomy, or urostomy. A colostomy or ileostomy involves attaching a section of the colon or small intestine respectively to an opening in the abdominal wall. A urostomy involves attaching a section of the urinary system to the abdominal wall in cases where drainage of urine through the bladder and urethra is not possible. In each case, a stoma for the excretion of waste from the body is formed at the opening that protrudes from the abdominal wall.

An ostomy appliance generally comprises a skin barrier or ostomy body fitment with a stomal aperture and a pouch for the collection of excreted waste. The ostomy body fitment of the ostomy appliance may be affixed by adhesive to the body of an individual around the stoma for the collection of excreted waste from the stoma into the pouch. An ostomy appliance may be provided as a single piece comprising both the ostomy body fitment and the pouch in a single pouch system. Alternatively, the ostomy appliance may be provided as a multi-piece (e.g., two piece) pouch system in which the ostomy body fitment and the ostomy pouch are separate components and may be attached together. For a multi-piece system, the ostomy body fitment may be first attached to the body of the ostomate around the stoma and the pouch may be attached to the ostomy body fitment to form the ostomy appliance for the collection of waste. Regardless of the system, it is important that an effective seal is formed between the stoma and the ostomy appliance to prevent leakage of excreted waste material.

Since ostomy appliances are often fitted by the patient themselves, it is also important to provide a means for users to attach the ostomy appliance to their body easily, securely, and reliably, and without discomfort. Furthermore, since ostomy appliances are unsightly, it is important to try and ensure such devices are as discrete as possible.

Reference is made to EP1378219 which describes an ostomy appliance incorporating a shapeable adhesive which can be manipulated to form a custom shaped fit around a wearer's stoma.

Reference is also made to FIGS. 1 to 3 which depict existing skin barriers or ostomy body fitments 1 for use in providing a seal between the user and an ostomy appliance comprising a tri-laminate structure 3 with a centrally located stomal aperture 5. The tri-laminate structure comprises a first, skin-facing, depicted lower, layer 7 comprising an adhesive for attaching the device to the skin of a patient, a second, clastic mid-layer 9 with a degree of memory for rebounding and hugging the stoma, and a third, pouch facing, depicted upper layer 11 comprising an adhesive hydrocolloid material operable to swell and hug the stoma, when in use. A removable release layer or liner (not shown) may be attached to the skin-facing layer 7 and also to the pouch-facing layer 11 to protect the respective layers until installation of the device by a user.

The ostomy body fitment 1 is intended to be installed according to a procedure comprising multiple, sequential operational steps. The operational steps include removing the release layer from the pouch-facing layer 11 and as shown in FIG. 2, rolling the tri-laminate structure 3 from the inner edge 13 of the stomal aperture 5 outwardly and upwardly to form a raised collar or “turtleneck” 15. As shown in FIG. 3 the device is affixed to the skin of the patient such that the stoma 17 extends through the stomal aperture 5 of the body fitment 1 and the rolled collar 15 extends upwardly from the pouch-facing layer 11 and hugs the external wall of the stoma 17 with a snug fit. An ostomy pouch (not shown) may then be adhered to the ostomy body fitment 1, via the adhesive surface of the pouch-facing layer 11 such that a secure and reliable seal is formed between the stoma 17 and the resulting ostomy appliance.

One problem with existing ostomy body fitments as described above is that the multi-step application procedure is difficult for users to follow, even with accompanying instructions. Consequently, users frequently remove the wrong release layer at the wrong time and/or roll the device in the wrong direction. This can result in the device being incorrectly applied such that the collar extends inwardly toward the user and/or such that the wrong adhesive side is affixed to the skin of the user. The incorrect application of the ostomy body fitment may compromise the effectiveness of the seal between the ostomate and an ostomy appliance, and thereby give rise to undesirable leakage from the stoma, as well as discomfort.

A further problem is that the material of the pouch-facing and skin-facing adhesive layers can have an undesirable or unsightly colour e.g., yellow. This can cause the ostomy body fitment to be too conspicuous when applied to the skin of the ostomate and give rise to an unsightly appearance, particularly when the upper torso of the ostomate is at least partially exposed e.g., when swimming.

The present invention seeks to provide an improved ostomy body fitment that at least mitigates the above problems.

### SUMMARY OF THE INVENTION

The present invention provides an ostomy body fitment according to the appended claims.

The present disclosure provides an ostomy body fitment. The ostomy body fitment may be suitable for forming a seal between an ostomy appliance and a stoma of an ostomate. The ostomy body fitment may comprise an adhesive layer. The ostomy body fitment may comprise a substrate layer. The ostomy body fitment may comprise a pair of adhesive layers. The substrate layer may be arranged between the adhesive layers. In one embodiment, the substrate layer may be a different colour from one, or each, adhesive layer.

An adhesive layer of the pair of adhesive layers may have a first hue. Both adhesive layers may have the first hue. The substrate layer may have a second hue. The colour of the first hue may be different from the colour of the second hue. In one embodiment, the colour of the substrate layer may contrast with the colour of the adhesive layer/layers. In one embodiment, the colour of the first hue may be complementary to the colour of the second hue.

At least one adhesive layer, for example at least one of the pair of adhesive layers may be translucent. A surface of the substrate layer may comprise an image arranged to be visible through a translucent adhesive layer (e.g., through one of the pair of adhesive layers).

The ostomy body fitment may be for attaching an ostomy appliance to a person's body.

Complementary colours are colours which, when combined or mixed, cancel each other out (i.e., lose hue) by producing a grayscale colour like white or black. Complementary colours are also known as “opposite colours”, on the basis that they are opposite each other on the colour wheel, e.g., on the RGB (red, green, blue) colour wheel.

Advantageously, the layers with hues of complementary colours neutralise one another such that the overall colour of the ostomy body fitment is more neutral in appearance. By combining layers with hues of complementary colours it is possible to produce an ostomy body fitment that is less dominated by a particular colour, e.g., yellow, of one or more layers and, therefore, more inconspicuous when applied to the body of an ostomate.

According to a first embodiment of the invention, there is provided an ostomy body fitment for forming a seal between an ostomy appliance and a stoma of an ostomate comprising an adhesive layer and a substrate layer, wherein the adhesive layer has a first hue and the substrate layer has a second hue, and wherein the colour of the first hue is different from the colour of the second hue.

Preferably the colour of the first hue is complementary to the colour of the second hue. Accordingly, a preferred embodiment of the invention provides an ostomy body fitment for forming a seal between an ostomy appliance and a stoma of an ostomate comprising an adhesive layer and a substrate layer, wherein the adhesive layer has a first hue and the substrate layer has a second hue, and wherein the colour of the first hue is complementary to the colour of the second hue.

Preferably the substrate layer is arranged between the adhesive layer and another adhesive layer (which preferably has the same first hue) i.e. between a pair of adhesive layers. Accordingly, a preferred embodiment of the invention provides an ostomy body fitment for forming a seal between an ostomy appliance and a stoma of an ostomate comprising a pair of adhesive layers and a substrate layer arranged between the adhesive layers, wherein the adhesive layers each have a first hue and the substrate layer has a second hue, and wherein the colour of the first hue is different from the colour of the second hue.

According to another a preferred embodiment there is provided an ostomy body fitment for forming a seal between an ostomy appliance and a stoma of an ostomate comprising a pair of adhesive layers and a substrate layer arranged between the adhesive layers, wherein the adhesive layers each have a first hue and the substrate layer has a second hue, and wherein the colour of the first hue is complementary to the colour of the second hue.

The first hue may be from a yellow region of a colour space. The second hue may be from a blue region of the colour space. In another embodiment, the second hue may be from a region of the colour space that corresponds to magenta or Bordeaux in colour. The colour space may be the CIELAB colour space. The first hue may have a positive b* value of the CIELAB colour space and the second hue may have a negative b* value of the CIELAB colour space. The b* value of the first hue and the b* value of the second hue may be substantially equal and opposite. The b* value of the first hue and the b* value of the second hue may have a greater magnitude than their respective a* values. The magnitude of the b* value of the first hue may be within 30%, more preferably 20%, more preferably 10%, and more preferably 5%, of the magnitude of the second hue. The colour coordinates of the first hue in a colour space may be substantially equal and opposite to the colour coordinates of the second hue in the colour space. The substrate layer may be tinted to achieve the second hue.

The pair of adhesive layers and the substrate layer may be coextensive. The ostomy body fitment may comprise an annular formation. The ostomy body fitment may be disc shaped. The ostomy body fitment may have a generally planar configuration. Alternatively, the ostomy body fitment may have a convex contour.

At least one (and optionally each) of the adhesive layers may be translucent. The substrate layer may be translucent. In another embodiment, the substrate layer may be opaque. A surface of the substrate layer may comprise an image visible through a translucent adhesive layer of the pair of adhesive layers.

Accordingly, a second aspect of the invention provides an ostomy body fitment for forming a seal between an ostomy appliance and a stoma of an ostomate comprising a pair of adhesive layers and a substrate layer arranged between the pair of adhesive layers, wherein at least one of the pair of adhesive layers is translucent, and wherein a surface of the substrate layer comprises an image arranged to be visible through a translucent adhesive layer of the pair of adhesive layers.

Such an arrangement can be beneficial in terms of allowing instructions for use and so forth to be incorporated into the body fitment itself by means of the image. This can not only make it easier for the ostomate to understand the instructions, but also more likely that the ostomate will consider the instructions. At present it is considered that a barrier to use of certain ostomy body fitments (in particular those that make up one part of a two-part ostomy pouch system and are intended to be adhered on one side to the user's skin and on the other side subsequently adhered to a pouch) is that users do not understand, or do not even read, the instructions that are provided in the packages in which body fitments are supplied. This can, for example, lead to the wrong side of the body fitment being adhered to the user's body, or the fitment being rolled in the wrong direction, so that it does not seal well to the user's body.

Of course, the ostomy body fitment of the first aspect of the invention may have any of the optional features set out above.

The image may relate to one or more procedural steps in the utilization of the ostomy body fitment. The image on the substrate layer may be darker than the substrate layer. The image on the substrate layer may have an L* value in the CIELAB colour space that is lower than the L* value of the substrate layer. The image on the substrate layer may be black. The image on the substrate layer may comprise one or more of numbers, letters, pictorial representations, and patterns. The image on the substrate layer may comprise branding. The image on the substrate layer may comprise text and/or logo in a direction that is forward when viewed from a first side of the ostomy body fitment, e.g., the skin-facing side, and mirrored when viewed from the opposite side of the ostomy body fitment, e.g., the pouch-facing side.

The substrate layer may comprise an ink absorbent material. The substrate layer may comprise a sheet of polymer material. The substrate layer may comprise polyurethane or polyethylene material.

A stomal aperture may extend through the adhesive layers and the substrate layer. The stomal aperture may be formed through a centre of the ostomy body fitment. The stomal aperture may be circular.

One adhesive layer of the pair of adhesive layers may comprise a first adhesive for affixing the ostomy body fitment to a user's skin and the other adhesive layer may comprise a second adhesive for affixing the ostomy body fitment to an ostomy pouch. The first adhesive and the second adhesive may comprise the same adhesive. An image may be printed on a surface of the substrate layer underlying the adhesive layer for affixing the ostomy body fitment to a user's skin. The image may comprise indicia relating to the application of the associated side of the ostomy body fitment to the skin of an ostomate.

A release layer may be attached to each adhesive layer. Each release layer may comprise an image relating to the order in which the release layers are intended to be removed from the ostomy body fitment when in use.

The invention may be applied to so-called one-piece appliances in which the ostomy body fitment is incorporated into an ostomy appliance such that the body fitment is not intended to be separable from the ostomy pouch. Accordingly, in one embodiment there is provided a one-piece ostomy appliance comprising a pouch and a body fitment according as disclosed, or according to the first or second aspect of the invention, optionally including any optional feature outlined above. The invention may also be applied to so-called two-(or more) piece appliances in which the ostomy body fitment and ostomy pouch are removably attachable. Accordingly, in another embodiment there is provided a body fitment as disclosed, or according to the first or second aspect of the invention, optionally including any optional feature outlined above, comprising a fastening means for fastening the body fitment to a pouch. The fastening means may for example be a coupling, such as a clip for releasably coupling the body fitment to a pouch.

In a third aspect, the present invention provides a method of forming a seal between an ostomy appliance and a stoma of an ostomate comprising utilizing an ostomy body fitment according to the first aspect or the second aspect of the invention.

The method may comprise the step of attaching a pouch of the ostomy appliance to the ostomy body fitment of the ostomy appliance. The pouch may be attached to the ostomy body fitment after the ostomy body fitment is affixed to the body of an ostomate.

In a fourth aspect, the present invention provides an ostomy appliance comprising an ostomy body fitment according to the first aspect or the second aspect of the invention. The ostomy appliance may further comprise an ostomy pouch. The ostomy pouch and the ostomy body fitment may be releasably attachable.

In a fifth aspect, the present invention provides a method of manufacturing an ostomy body fitment comprising the steps of:
providing an adhesive layer having a first hue (preferably providing a pair of adhesive layers each having a first hue);
providing a substrate layer having a second hue;
wherein the colour of the first hue is complementary to the colour of the second hue; and
affixing the substrate layer to a surface of the adhesive layer (preferably to a surface of each adhesive layer such that the substrate layer is between the pair of adhesive layers).

In a sixth aspect, the present invention provides a method of manufacturing an ostomy body fitment comprising the steps of:
providing a sheet of material as a substrate layer;
applying an image to a first surface of the substrate layer; and
affixing a first adhesive layer to the first surface of the substrate layer to overlay the image.

The step of applying an image to the surface of the substrate layer may comprise printing or painting the image on the surface.

The step of affixing the adhesive layer to the substrate layer may comprise hot rolling. The method may further comprise the step of applying an image to a second surface of the substrate layer, opposite the first surface. The method may further comprise the step of affixing a second adhesive layer to the second surface of the substrate layer. The method may comprise the step of heating or coating the image on the first surface of the substrate layer and/or the image on the second surface of the substrate layer prior to affixing the first or second adhesive layers, respectively. The first surface of the substrate layer may be intended to be the skin-facing surface. The or each image may comprise indicia relating to the application of the ostomy body fitment.

The skilled person will appreciate that except where mutually exclusive, a feature described in relation to any one of the aspects, embodiments or examples described herein may be applied to any other aspect, embodiment, or example. Furthermore, except where mutually exclusive, any feature described herein may be applied to any aspect and/or combined with any other feature described herein.

### BRIEF DESCRIPTION OF THE DRAWINGS

In order that the invention may be more clearly understood one or more embodiments thereof will now be described, by way of example only, with reference to the accompanying drawings, of which:

FIG. 1 shows a perspective view of a schematic representation of an existing ostomy body fitment;

FIG. 2 shows a perspective view of a render of an existing ostomy body fitment being manipulated by a user to shape a stomal aperture;

FIG. 3 shows the ostomy body fitment of FIG. 2 when applied to the body of an ostomate such that the stoma of the ostomate extends through the stomal aperture;

FIG. 4 shows a perspective view of a schematic representation of an ostomy body fitment according to an embodiment of the present invention;

FIG. 5 shows an enlarged partial view of a tri-laminate structure from which the ostomy body fitment shown in FIG. 4 may be formed, with the three layers separated at a corner;

FIG. 6 shows a perspective view of a schematic representation of an ostomy body fitment according to another embodiment of the present invention;

FIG. 7 shows an enlarged partial view of a tri-laminate structure from which the ostomy body fitment shown in FIG. 6 may be formed, with the three layers separated at a corner;

FIG. 8 shows an enlarged partial view of an alternative tri-laminate structure from which the ostomy body fitment shown in FIG. 6 may be formed, with the three layers separated at a corner;

FIG. 9 shows a top plan view of a schematic representation of an ostomy body fitment according to another embodiment of the present invention;

FIG. 10 shows an underside view of the ostomy body fitment shown in FIG. 9; and

FIG. 11 shows a side cross sectional view of a schematic representation of an ostomy body fitment according to an embodiment of the present invention when applied to an ostomate and surrounding the stoma of the ostomate.

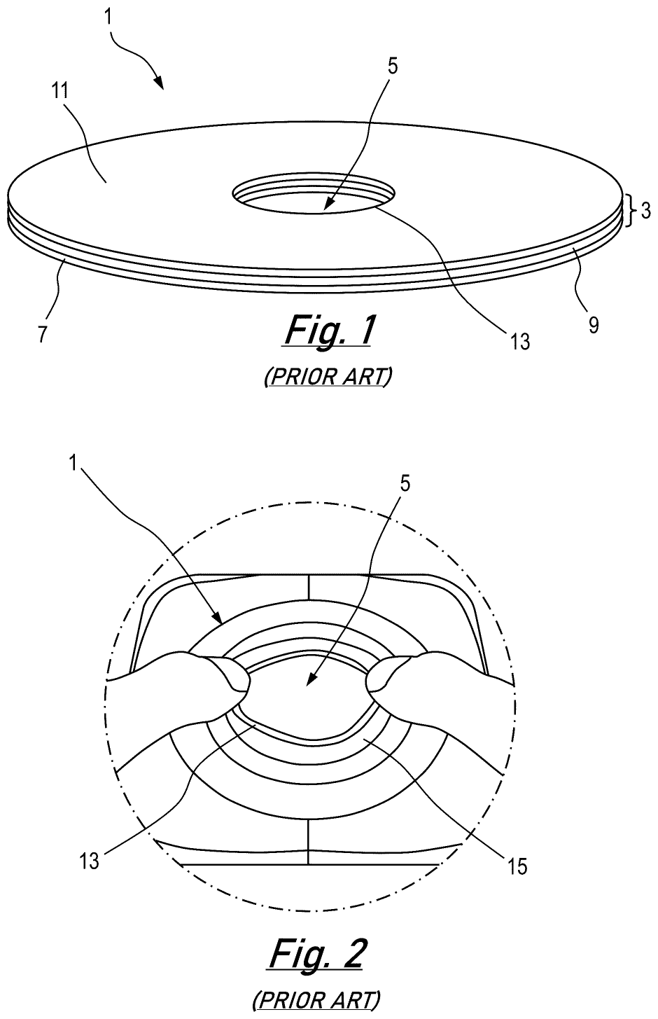

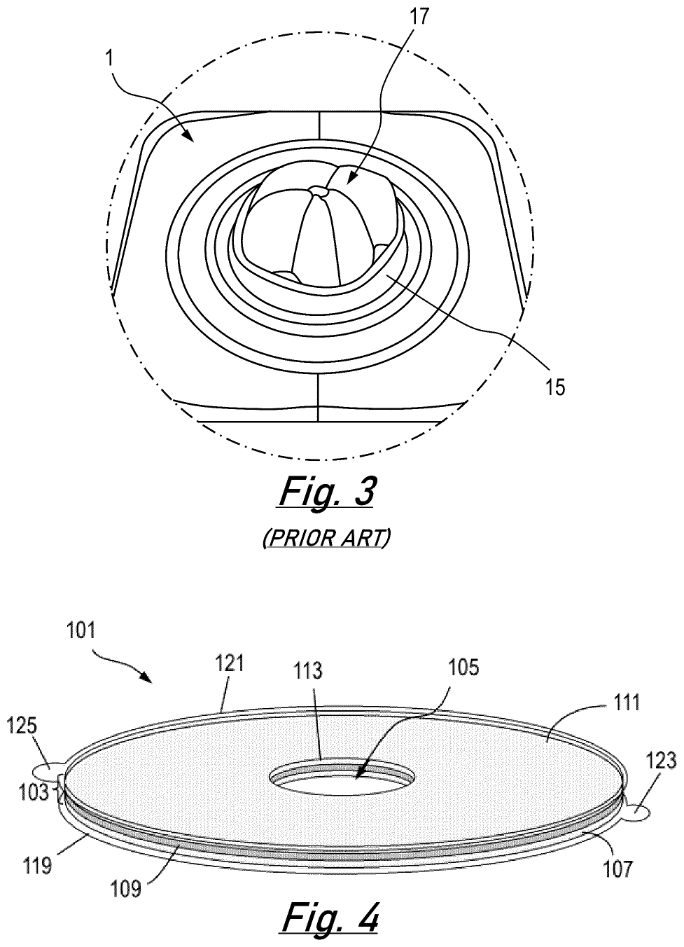

### DETAILED DESCRIPTION OF THE INVENTION

In the following detailed description, numerous specific details are set forth in order to provide a thorough understanding of various embodiments and the inventive concept. However, those skilled in the art will understand that the present invention may be practiced without these specific details or with known equivalents of these specific details, that the present invention is not limited to the described embodiments, and that the present invention may be practiced in a variety of alternative embodiments. It will also be appreciated that well known methods, procedures, components, and systems may have not been described in detail.

In the following description, reference to “skin-facing” and “pouch-facing” should be taken to be in relation to the body of a user or “ostomate” of an ostomy body fitment, unless otherwise stated. For example, a skin-facing layer should be taken to be the layer intended to be affixed or attached to the skin of the ostomate, and a pouch-facing layer should be taken to be the layer intended to face away from the body of the ostomate when the skin-facing layer is affixed to the skin of the ostomate.

FIG. 4 depicts an ostomy body fitment 101 of a two-piece ostomy pouch system for forming a seal between a stoma (not shown) and an ostomy pouch (not shown). The ostomy body fitment 101 comprises a tri-laminate structure 103. The tri-laminate structure 103 defines a stomal aperture 105 with an inner edge 113 for the receipt of a stoma 117 of an ostomate. The stomal aperture 105 is generally circular in its initial, undeformed state so as to substantially correspond to the generally circular outer wall of a stoma. The stomal aperture 105 may have an initial diameter of between 10 mm and 100 mm, preferably between 20 mm and 60 mm. Preferably, the stomal aperture 105 is deformable from a first diameter to a second diameter, and preferably by approximately 10 mm. In one form, the initial diameter of the stomal aperture 105 is generally about 20 mm and deformable up to a diameter of about 30 mm. In another form, the initial diameter of the stomal aperture 105 is generally about 30 mm and deformable up to a diameter of about 40 mm. In another form, the initial diameter of the stomal aperture 105 is generally about 40 mm and deformable up to a diameter of about 50 mm.

The tri-laminate structure 103 comprises a skin-facing adhesive layer 107, a middle substrate layer 109, and a pouch-facing adhesive layer 111. The skin-facing adhesive layer 107 and/or pouch-facing adhesive layer 111 are made from a pliable and/or mouldable and/or shapeable adhesive. For example, the two adhesive layers 107, 111 may comprises a medical grade adhesive such as a hypoallergenic hydrocolloid. Examples of suitable adhesives are those produced by ConvaTec under the brand names Stomahesive® and Durahesive®. Such adhesives are generally tacky, putty-like, and substantially non-elastic, with low memory. The choice of adhesive may depend on the intended purpose of the ostomy appliance and its duration of application. For example, where the ostomy appliance comprises a closed bag or pouch intended for short wear or an open bag or pouch intended for short/medium wear, the skin facing adhesive layer 107 may comprise Stomahesive® and the pouch facing adhesive layer 111 may comprise Durahesive®. Where the ostomy appliance is intended to be used by a patient with an ileostomy or urostomy for medium/long wear, both adhesive layers 107, 111 may comprise the more durable Durahesive®. At least one and preferably both adhesive layers 107, 111 are translucent so that the substrate layer 109 is visible from either or both sides of the ostomy body fitment 101.

The skin-facing and pouch-facing adhesive layers 107, 111 are each between around 0.3 mm and 1.5 mm in thickness. The middle substrate layer 109 is between around 0.2 mm and 1.0 mm in thickness and is thinner than the individual adhesive layers 107, 111. In the embodiment shown, the skin-facing adhesive layer 107 is 0.38 mm in thickness, the substrate layer 109 is 0.2 mm in thickness, and the pouch-facing adhesive layer is 1.14 mm in thickness. The pair of adhesive layers 107, 111 and the substrate layer 109 have a substantially uniform thickness throughout and are substantially coextensive i.e., they each extend across substantially the same area. In the embodiment shown, the three layers 107, 109, 111 are substantially disc shaped and each has an outer diameter of approximately 100 mm. The skilled person will appreciate that one or more of the three layers 107, 109, 111 may have a variable thickness across their respective diameters so as to create regions of different material characteristics e.g., having varying rigidity or surface shape.

In the embodiment shown in FIG. 4, each adhesive layer 107, 111 is covered by a release layer 119, 121 in the form of a thin, circular sheet made from, or coated by, a suitable material such as silicone to facilitate removal of the release layer 119, 121 from the corresponding adhesive layer 107, 111. Each release layer 119, 121 has substantially the same outer shape as the corresponding adhesive layer 107, 111 but in the present embodiment extends slightly beyond the outer boundaries of the adhesive layers 107, 111 and serves to protect the adhesive layers 107, 111 until the ostomy body fitment 101 is utilized by an ostomate. Each release layer 119, 121 comprises a small tab 123, 125 which protrudes from the outer edge of the corresponding release layer 119, 121 to allow the release layers 119, 121 to be easily grasped by a user of the ostomy body fitment 101 and removed from the corresponding adhesive layers 107, 111 when being applied to an ostomate.

Whilst in the embodiment shown a single release layer 119, 121 is provided to protect a corresponding adhesive layer 107, 111, it will be appreciated that more than one release layer 119, 121 may be provided per adhesive layer 107, 111. For example, two or more release layers may be provided that individually partially cover different regions of a corresponding adhesive layer 107, 111 and collectively cover the entire surface of the adhesive layer 107, 111. Advantageously, different regions of an adhesive layer 107, 111 may be exposed at different times so that one or more operational steps may be performed on part of the ostomy body fitment 101 whilst protecting other parts until a subsequent operational step in the installation of the ostomy body fitment 101.

Referring once more to FIG. 4, the two adhesive layers 107, 111 have a first hue from a colour space and the substrate layer 109 has a second hue from the colour space. The colour space may be the 3-dimensional CIELAB colour space built-up from three axes that are perpendicular to one another, namely the L*-axis which describes the achromatic colours i.e., the “lightness”, and the a*-axis and the b*-axis which describe the chromatic or “real” colours. In the present embodiment, the two adhesive layers 107, 111 have colour coordinates that fall within the yellow region of the CIELAB colour space i.e., a positive b* value which is greater in magnitude than the corresponding a* value. For example, the b* value may be +70 and the a* value may be −10. Correspondingly, the substrate layer 109 has colour coordinates that fall within the blue region of the CIELAB colour space i.e., a negative b* value which is greater in magnitude than the corresponding a* value. Accordingly, the substrate layer 109 may have a colour which is approximately complementary to the colour of the two adhesive layers 107, 111. For example, based on the above CIELAB colour coordinates for the adhesive layers 107, 111, the substrate layer 109 may have a b* value of −70 and an a* value of +10. Preferably, the colour coordinates of the hue of the substrate layer 109 are substantially equal and opposite to the colour coordinates of the hue of the adhesive layers 107, 111. However, there may be a degree of tolerance in the choice of the two hues and it may be acceptable to select two hues with respective colour coordinates that are not precisely opposite in the colour space but are sufficiently complementary to achieve a more neutral colour when combined.

The substrate layer 109 comprises a sheet of polymer material such as polyurethane which is clastic and has a degree of memory for rebounding and hugging the stoma 117. The substrate layer 109 may also be translucent such that light can pass through the adhesive layers 107, 111 and the substrate layer 109 from one side of the ostomy body fitment 101 to the other. Accordingly, a pouch-facing surface of the substrate layer 109 may be visible through the skin-facing adhesive layer 107 and, conversely, a skin-facing surface of the substrate layer 109 may be visible through the pouch-facing adhesive layer 111. With particular reference to FIG. 5 the substrate layer 109 is tinted to achieve the desired blue hue that complements the yellow hue of the two adhesive layers 107, 111. By selecting the appropriate blue hue for the substrate layer 109 that complements the yellow hue of the two adhesive layers 107, 111, it is possible to counter the yellow hue of the two adhesive layers 107, 111 and thereby achieve a more neutral, whiter appearance that is more subtle and less unsightly when the resulting ostomy body fitment 101 is applied to the body of an ostomate.

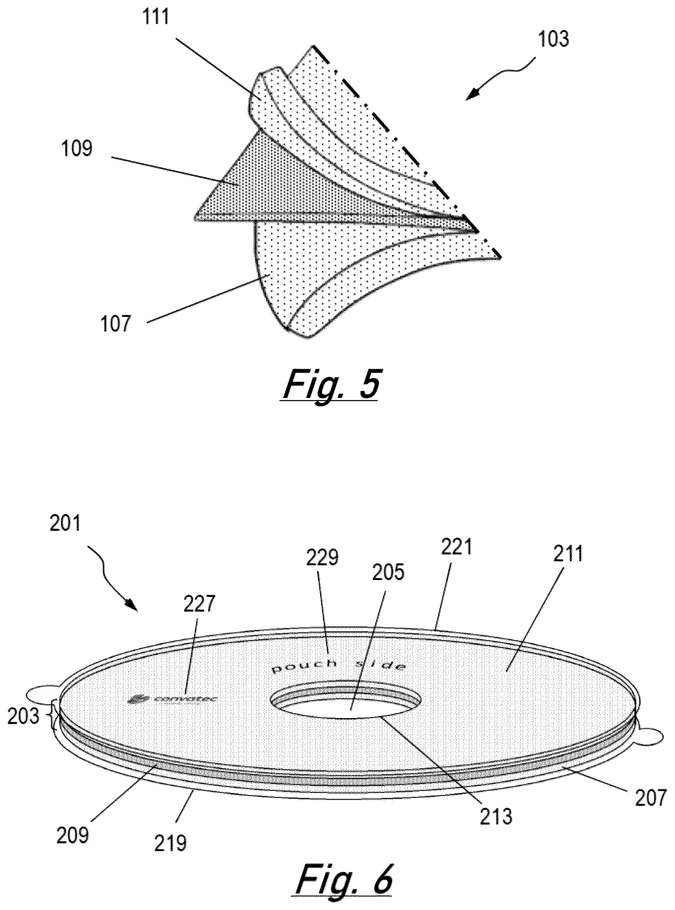

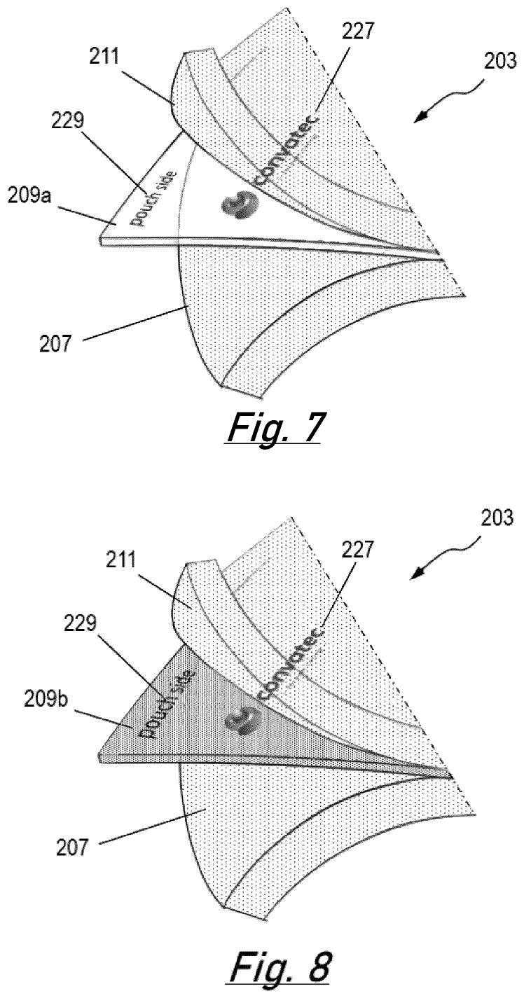

Referring to FIG. 6, in an alternative embodiment, the ostomy body fitment 201 comprises one or more images 227, 229 formed on a surface of the substrate layer 209. Accordingly, the material of the substrate layer 209 may be further selected according to its ability to receive and retain an ink upon a surface thereof and thereby comprise a polymer material with ink absorbent properties. The images 227, 229 may be painted or printed onto the skin-facing and/or pouch-facing surfaces of the substrate layer 209 using any suitable painting or printing technique or combination of painting and printing techniques.

As used herein, an “image” may comprise one or more of numbers, letters, pictorial representations, and patterns, and may be created by one or more coloured mediums that may have multiple colours or contrasting textures. For example, in the embodiment shown in FIG. 6, one image 227 may comprise a company logo or other company branding. Another image 229 may comprise writing or a pictorial representation that relates to the correct usage of the ostomy body fitment 201. For example, an image 229 may comprise the words “pouch side” to indicate to the user which side of the ostomy body fitment 201 is intended face away from the skin of the ostomate for the attachment of an ostomy pouch. An image 227, 229 could also comprise a repeating pattern that may be specific to the brand of the ostomy body fitment 201. A repeating pattern may therefore serve to identify the ostomy body fitment 201 as originating from a particular brand and/or may help to visually break up the surface of the ostomy body fitment 201. It will be appreciated that any number of images 227, 229 may be applied to the substrate layer 209 as desired, subject to the available space, and also that one or more images 227, 229 may be applied to both the pouch-facing and skin-facing surfaces of the substrate layer 209.

As shown in FIG. 7, the ostomy body fitment 201 may comprise a tri-laminate structure 203 in which the substrate layer 209a is colourless or a colour that is different from the colour of the two adhesive layers 207, 211, e.g., magenta or Bordeaux. As shown in FIG. 8, the substrate layer 209b may be coloured or tinted so as to complement the colour of the adhesive layers 207, 211 and/or to enhance the contrast with the images 227, 229 of the substrate layer 209. In either case, the images 227, 229 on the substrate layer 209 are preferably provided in a colour that contrasts with the colour of the adhesive layers 207, 211 so that the images 227, 229 are clearly visible through the adhesive layers 207, 211.

Furthermore, the substrate layer 209 may be opaque so that only the skin-facing surface of the substrate layer 209 is visible through adhesive layer 207 from the skin-facing side of the ostomy body fitment 201 and, conversely, so that only the pouch-facing side of the substrate layer 209 is visible through adhesive layer 211 from the pouch-facing side of the ostomy body fitment 201. Advantageously, this allows one or more images 227, 229 to be printed on each side of the substrate layer 209 without affecting the legibility or appearance of two diametrically opposed images 227, 229. For example, one image may comprise indicia relating to the side of the ostomy body fitment 201 that should be affixed to the skin of an ostomate e.g., “This side toward skin”, and hence be printed on the skin-facing surface of the substrate layer 209. Another image may comprise a company logo or pattern and be printed on the pouch-facing surface of the substrate layer 209. By using an opaque substrate layer 209, the two images printed on opposing surfaces of the substrate layer 209 will not clash and, hence, be clearly legible through the corresponding, adjacent adhesive layers 207, 211, notwithstanding that they may be diametrically opposed and otherwise overlapping.

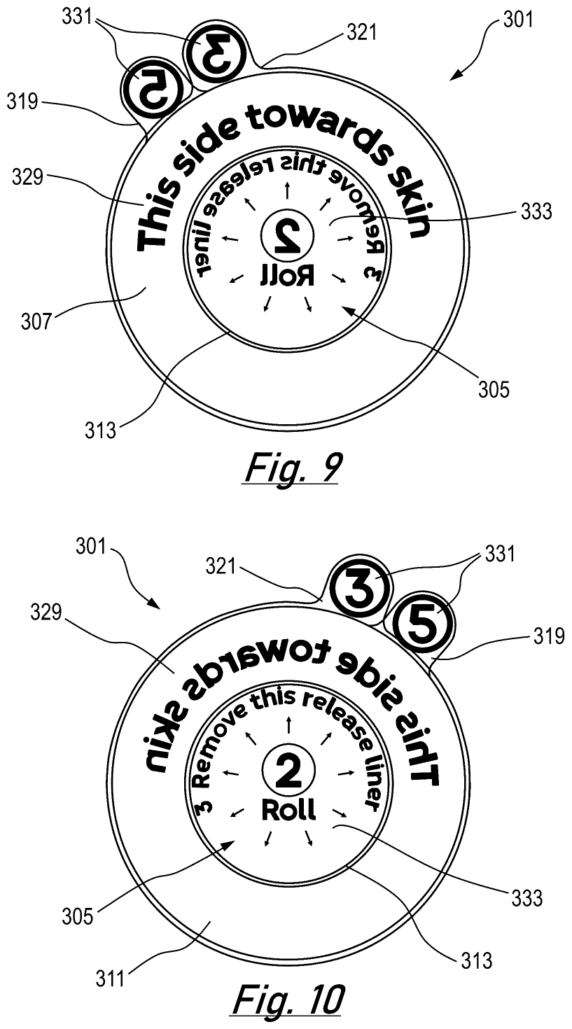

Referring to FIGS. 9 and 10, in an alternative embodiment, one or more images 329 may be provided on the substrate layer 309 and one or more images 331, 333 may be provided on the release layers 319, 321. The images 329, 331, 333 on the substrate layer 309 and the release layers 319, 321 respectively may comprise instructions relating to the correct sequence in which to install the ostomy body fitment 301 to the body of the ostomate. For example, a release layer image 331, 333 may consist of or comprise a number that represents the order in which to carry out a procedural step such as the removal of a release layer 319, 321 or the rolling of the tri-laminate structure 303. A release layer image 333 may alternatively or additionally comprise writing and/or a pictorial representation associated with the corresponding step number to indicate to the user the action to perform when that step of the installation procedure is reached. Providing written instructions may further encourage the correct usage of the ostomy body fitment 301 by indicating to the user the correct side to affix the ostomy body fitment 301 to the ostomate based on the side of the ostomy body fitment 301 viewed from which the written instruction proceeds in the correct direction.

Whilst the above embodiments describe and depict a disc shaped ostomy body fitment it will be appreciated that the ostomy body fitment and corresponding release layers may have a different shape such as square, star, rectangle, or oval, provided there is a surface through which a stomal aperture of appropriate size may be defined and expanded, and also provided there is sufficient surface area to ensure good contact with the body of an ostomate. Furthermore, whilst the above embodiments described and depict an ostomy body fitment that is substantially planar, it will be appreciated that part or all of the ostomy body fitment may comprise a convex or concave contour. This may be desirable when used on an ostomate with a retracted stoma or a stoma that does not sufficiently protrude from the abdominal wall to extend through the stomal aperture of a planar ostomy body fitment. The convexity of the ostomy body fitment may serve to push against the abdominal wall of an ostomate and urge the stoma into a more protruded position for the capture of the stomal aperture. In such an arrangement clarity to the user in terms of which side to apply to the skin is even more important.

Regardless of the embodiment, the ostomy body fitment 101, 201, 301 is installed according to a process comprising multiple sequential steps. In a first step, the appropriate release layer 121, 221, 321 is removed from the pouch-facing adhesive layer 111, 211, 311 to expose the inner edge 113, 213, 313 of the stomal aperture 105, 205, 305 and at least a portion of the pouch-facing surface of the adhesive layer 111, 211, 311 extending radially from the inner edge 113, 213, 313.

In a second step, a user urges the inner edge 113, 213, 313 of the stomal aperture 105, 205, 305 upwardly and outwardly so as to roll the ostomy body fitment 101, 201, 301 upon itself along the pouch-facing surface of the pouch-facing adhesive layer 111, 211, 311 and thereby form a raised collar 115, 215, 315 extending around the circumference of the stomal aperture 105, 205, 305. The deforming and/or rolling action allows the user to increase the diameter of the stomal aperture 105, 205, 305 so as to approximately correspond to the diameter of the target stoma 117, 217, 317.

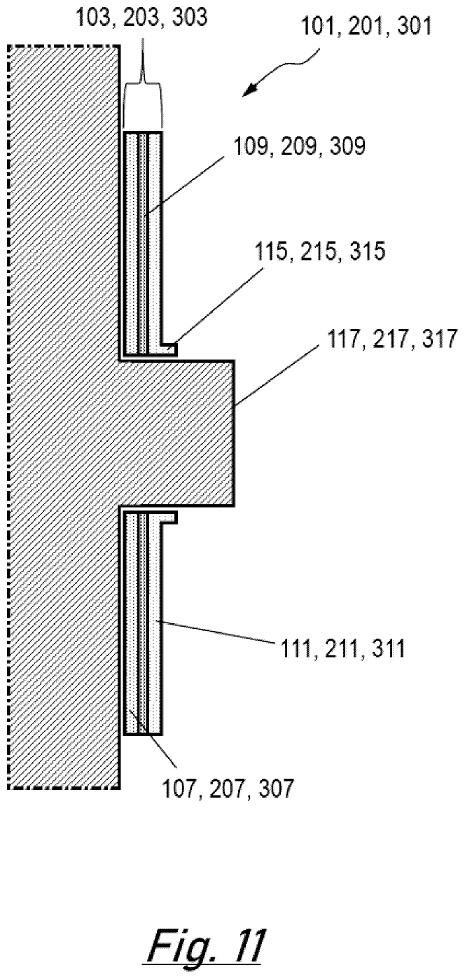

When the user has achieved a stomal aperture diameter that corresponds to or is slightly greater than the diameter of the target stoma 117, 217, 317, in a third step, the user removes the release layer 119, 219, 319 from the skin-facing adhesive layer 107, 207, 307 and affixes the ostomy body fitment 101, 201, 301 to the skin of the ostomate such that the stoma 117, 217, 317 extends through the stomal aperture 105, 205, 305. As shown in FIG. 11, in this position, the ostomy body fitment 101, 201, 301 encircles the stoma 117, 217, 317 and the collar 115, 215, 315 formed in the previous step extends upwardly along the outer wall of the stoma 117, 217, 317 away from the body of the ostomate. Due to the degree of memory of the substrate layer 109, 209, 309, the ostomy body fitment 101, 201, 301 rebounds such that the stomal aperture 105, 205, 305 decreases in diameter and the collar 115, 215, 315 hugs the stoma 117, 217, 317 with a snug fit.

In a fourth step, the user affixes an ostomy pouch (not shown) to the ostomy body fitment so as to form an ostomy appliance. Accordingly, a seal is formed between the ostomy appliance and the stoma such that waste is excreted into the pouch from the stoma without leakage.

The one or more embodiments are described above by way of example only. Many variations are possible without departing from the scope of protection afforded by the appended claims.

1. An ostomy body fitment for forming a seal between an ostomy appliance and a stoma of an ostomate comprising an adhesive layer and a substrate layer, wherein the adhesive layer has a first hue and the substrate layer has a second hue, and wherein the colour of the first hue is different from the colour of the second hue.

2. An ostomy body fitment as claimed in claim 1 wherein the colour of the first hue is complementary to the colour of the second hue.

3. An ostomy body fitment as claimed in claim 1 or claim 2 comprising a pair of adhesive layers, wherein the substrate layer is arranged between the adhesive layers, and wherein the adhesive layers each have the first hue.

4. The ostomy body fitment as claimed in any preceding claim wherein the first hue is from a yellow region of a colour space, and the second hue is from a blue region of the colour space.

5. The ostomy body fitment as claimed in any preceding claim wherein the first hue has a positive b* value of the CIELAB colour space and the second hue has a negative b* value of the CIELAB colour space.

6. The ostomy body fitment as claimed in claim 5 wherein the b* value of the first hue and the b* value of the second hue are substantially equal and opposite.

7. The ostomy body fitment as claimed in claim 5 or claim 6 wherein the b* value of the first hue and the b* value of the second hue have a greater magnitude than their respective a* values.

8. The ostomy body fitment as claimed in any preceding claim wherein the substrate layer is tinted or coated to achieve the second hue.

9. The ostomy body fitment as claimed in any preceding claim comprising an annular formation.

10. The ostomy body fitment as claimed in any preceding claim wherein the ostomy body fitment is disc shaped.

11. The ostomy body fitment as claimed in any preceding claim comprising a generally planar configuration.

12. The ostomy body fitment as claimed in any one of claims 1 to 9 comprising a convex contour.

13. The ostomy body fitment as claimed in any preceding claim wherein at least one of the adhesive layers is translucent.

14. The ostomy body fitment as claimed in claim 13 wherein a surface of the substrate layer comprises an image visible through a translucent adhesive layer.

15. The ostomy body fitment as claimed in claim 14, wherein the image relates to one or more procedural steps in the utilization of the ostomy body fitment.

16. The ostomy body fitment as claimed in any preceding claim wherein the substrate layer comprises an ink absorbent material.

17. The ostomy body fitment as claimed in any preceding claim wherein the substrate layer comprises polyurethane material.

18. The ostomy body fitment as claimed in any preceding claim comprising a stomal aperture extending through the or each adhesive layer and the substrate layer.

19. The ostomy body fitment as claimed in claim 3 or any of claims 4-18 when dependent on claim 3 wherein one adhesive layer comprises a first adhesive surface for contacting the skin of an ostomate and the other adhesive layer comprises a second adhesive surface opposite the first adhesive surface.

20. The ostomy body fitment as claimed in any preceding claim wherein a removable release layer is attached to the or each adhesive layer.

21. The ostomy body fitment as claimed in claim 20 wherein one or more removable release layers comprises an image relating to a sequential order in which the release layers are intended to be removed from the ostomy body fitment when in use.

22. An ostomy appliance comprising an ostomy body fitment as claimed in any one of claims 1 to 21.

23. A method of manufacturing an ostomy body fitment comprising the steps of:
providing an adhesive layer having a first hue;
providing a substrate layer having a second hue;
wherein the colour of the first hue is different from the colour of the second hue; and
affixing the substrate layer to a surface of the adhesive layer.

24. The method as claimed in claim 22 comprising providing a pair of adhesive layers each having the first hue; and affixing the substrate layer to a surface of each adhesive layer such that the substrate layer is between the pair of adhesive layers; wherein the colour of the first hue is complementary to the colour of the second hue.

25. The method as claimed in claim 22 or 23, wherein the step of affixing the substrate layer to a surface of each adhesive layer comprises hot rolling.

26. An ostomy body fitment for forming a seal between an ostomy appliance and a stoma of an ostomate comprising a pair of adhesive layers and a substrate layer arranged between the pair of adhesive layers, wherein at least one of the pair of adhesive layers is translucent, and wherein a surface of the substrate layer comprises an image arranged to be visible through a translucent adhesive layer of the pair of adhesive layers.

27. The ostomy body fitment as claimed in claim 26 wherein the substrate layer comprises an ink absorbent material.

28. The ostomy body fitment as claimed in claim 26 or 27 wherein the substrate layer comprises a sheet of polymer material.

29. The ostomy body fitment as claimed in claim 28 wherein the polymer material comprises polyurethane.

30. The ostomy body fitment as claimed in any of claims 26 to 29 wherein the ostomy body fitment is disc shaped.

31. The ostomy body fitment as claimed in any of claims 26 to 30 wherein the ostomy body fitment is planar.

32. The ostomy body fitment as claimed in any of claims 26 to 29 comprising a convex contour.

33. The ostomy body fitment as claimed in any of claims 26 to 32 wherein the substrate layer is tinted to achieve a hue that is complementary to the hue of the pair of adhesive layers.

34. The ostomy body fitment as claimed in any of claims 26 to 33 wherein the hue of the substrate layer contrasts with at least one hue of the image.

35. The ostomy body fitment as claimed in any of claims 26 to 34 comprising a stomal aperture extending through the adhesive layers and the substrate layers.

36. The ostomy body fitment as claimed in claim 35 wherein the stomal aperture is circular.

37. The ostomy body fitment as claimed in claim 35 or 36 wherein the stomal aperture is formed through a centre of the ostomy body fitment.

38. The ostomy body fitment as claimed in any of claims 26 to 37 wherein the image comprises an instruction relating to the use of the ostomy body fitment.

39. The ostomy body fitment as claimed in any of claims 26 to 38 wherein the image comprises one or more of numbers, letters, pictorial representations, and patterns.

40. The ostomy body fitment as claimed in any of claims 26 to 39 wherein the image comprises branding.

41. The ostomy body fitment as claimed in any of claims 26 to 40 wherein the image comprises text and/or logo in a direction that is forward when viewed from a first side of the ostomy body fitment and mirrored when viewed from a second side of the ostomy body fitment opposite the first side.

42. A method of affixing an ostomy appliance to an ostomate comprising utilizing the ostomy body fitment as claimed in any of claims 26 to 41.

43. An ostomy appliance comprising an ostomy body fitment as claimed in any one of claims 26 to 41.

44. A method of manufacturing an ostomy body fitment comprising the steps of providing a sheet of material as a substrate layer;
applying an image to a first surface of the substrate layer; and
affixing a first adhesive layer to the first surface of the substrate layer to overlay the image.

45. The method as claimed in claim 44, wherein the step of applying the adhesive layer to the surface of the substrate layer comprises hot rolling.

46. The method as claimed in claim 44 or 45, further comprising the step of applying an image to a second surface of the substrate layer opposite the first surface.

47. The method as claimed in any of claims 44 to 46, further comprising the step of affixing a second adhesive layer to the second surface of the substrate layer opposite the first surface.
